(19) United States
(12) Patent Application Publication
(10) Pub. No.: US 2024/0084259 A1
(43) Pub. Date: Mar. 14, 2024

(54) STEM CELL-DERIVED MATURE CARDIOMYOCYTES AND CARDIOVASCULAR DISEASE MODEL USING SAME

(71) Applicant: KOREA UNIVERSITY RESEARCH AND BUSINESS FOUNDATION, sEOUL (KR)

(72) Inventors: Do-Sun LIM, Seoul (KR); Seung Cheol CHOI, Seoul (KR); Hyung Joon JOO, Seoul (KR); Jong-Ho KIM, Seoul (KR); Chi-Yeon PARK, Seoul (KR); Yongdoo PARK, Seoul (KR)

(73) Assignee: KOREA UNIVERSITY RESEARCH AND BUSINESS FOUNDATION, Seoul (KR)

(21) Appl. No.: 17/767,822

(22) PCT Filed: Nov. 13, 2019
(86) PCT No.: PCT/KR2019/015386

§ 371 Date: Apr. 8, 2022

(30) Foreign Application Priority Data
Oct. 10, 2019 (KR) 10-2019-0125458

(51) Int. Cl. C12N 5/077; C12N 5/00; G01N 33/50; G01N 33/68

(52) U.S. Cl. CPC C12N 5/0657; C12N 5/0018; G01N 33/5061; G01N 33/6887; C12N 2500/02; C12N 2500/38; C12N 2501/119; C12N 2503/02; G01N 2333/805

(57) ABSTRACT

The present invention relates to stem cell-derived mature cardiomyocytes and a cardiovascular disease model using same and, more specifically, to differentiation into mature ventricular cardiomyocytes by culturing stem cells in a medium containing FGF4 and ascorbic acid, and use of the differentiated mature ventricular cardiomyocytes as a cardiovascular disease cell model. The mature ventricular cardiomyocytes, obtained by culturing stem cells in a medium containing FGF4 and ascorbic acid and inducing the differentiation thereof, and cardiovascular disease cell model using same according to the present invention are very useful for screening for cardiovascular disease therapeutic agents and evaluation of the toxicity of new drugs.

### TECHNICAL FIELD

The present invention relates to stem-cell-derived mature cardiomyocytes and a cardiovascular disease model using the same, and more particularly to differentiation of stem cells into mature ventricular cardiomyocytes by culturing stem cells in a medium containing FGF4 and ascorbic acid, and the use of the differentiated mature ventricular cardiomyocytes for a cardiovascular disease cell model.

### BACKGROUND ART

Heart disease is one of the major causes of death in adults, and the prevalence and mortality rates of myocardial infarction and angina pectoris, which are caused by coronary artery disease of the heart, are much higher than for other diseases. The heart is one of the essential organs for toxicity evaluation, comparable to hepatotoxicity, not only in the development of therapeutic agents for arrhythmia and vascular diseases, but also in the testing of various toxic materials such as anticancer drugs and the like (I. Kola & J. Landis, Nature Reviews Drug Discovery 3:711, 2004).

For cardiovascular disease modeling and drug toxicity evaluation, studies have been conducted using, as cell models, rat neonatal cardiomyocytes (L. Li et al., Pflugers Arch. 448;146, 2004; E. Gabrielova et al., Physiol. Res. 64:79, 2015) or animal-cell-derived cardiomyocyte lineages (A. S. Streng et al., Exp. Mol. Pathol. 96:339, 2014; J. Zhang et al., Cell Physiol. Biochem. 39:491, 2016). However, since adult cardiomyocytes obtained from experimental animals such as mice, rats and the like are difficult to isolate and culture, many limitations are imposed on the use thereof for drug toxicity evaluation and new drug screening, and moreover, animal-derived cell lineages, such as C2C12, H9C2, and HL-1 cells, which are cell line models for the study of cardiomyocyte toxicity, differ from human adult cardiomyocytes with regard to the maturation state of sarcomere and electrophysiological characteristics (Y. Y. Zhou et al., Am. J. Physiol. Heart Circ. Physiol. 297:H429, 2000), as well as inter-species differences. In particular, because rabbits, dogs, and monkeys have different cardiac action potential waveforms and cardiac action potential intervals from humans, evaluation using experimental animals may introduce errors due to inter-species differences between humans and animals, so the evaluation of cardiotoxicity of drugs using human ventricular cardiomyocytes has been suggested as an alternative. However, human cardiomyocytes are limited in supply and also difficult to use, so it is necessary to develop a test method using cardiomyocytes derived from human pluripotent stem cells (K. Takasuna et al., J. Pharmacol. Toxicol. Methods. 83:42, 2017; Cardiovascular Pharmacology Test Methods Guide, 2016. 12 National Institute of Food and Drug Safety Evaluation).

Recently, studies to detect new biomarkers that show toxicity to doxorubicin treatment using cardiomyocytes differentiated from human pluripotent stem cells or to identify the mechanism by which cardiomyopathy occurs due to doxorubicin have been conducted (G. Holmgren et al., Toxicology 328:101, 2015; A. Maillet et al., Sci. Rep. 6:25333, 2016; H. K. Taymaz-Nikerel et al., Sci. Rep. 8:13672, 2018; K. M. McSweeney et al., Cell Death Discov. 5:102, 2019). A study has reported the detection of cardiac troponin T (cTnT) and fatty acid binding protein 3 (FABP3), which are myocardial infarction biomarkers, in a culture medium as a result of doxorubicin treatment using, as a model, cardiomyocytes derived from human pluripotent stem cells (H. Andersson et al., J. Biotechnol. 150:175, 2010).

Since most cases of human heart disease develop in adulthood, the use of mature cardiomyocytes for disease modeling and drug toxicity evaluation enables evaluation with improved efficacy and safety. Therefore, it is necessary to study the differentiation of immature cardiomyocytes into mature cardiomyocytes and differentiation by cardiomyocyte type. However, cardiomyocytes differentiated from human embryonic or induced pluripotent stem cells are heterogeneous, and mostly have immature cardiomyocyte characteristics, similar to those of embryonic or fetal cardiomyocytes, and moreover, atrial, ventricular and nodal cardiomyocyte types are mixed therewith (X. Yang et al., Circ. Res. 114:511, 2014). It has been reported that the structural characteristics of immature cardiomyocytes derived from human pluripotent stem cells are that the cell morphology is circular, sarcomere formation and arrangement are incomplete, T-tubules are not formed, and mitochondria are small and irregularly distributed, and also the electrophysiology and beating characteristics are different from those of a real heart, that the formation of ion channels is incomplete, and that reactivity to drugs is low (X. Yang et al., Circ. Res. 114:511, 2014).

It has been reported that the maturation state of cardiomyocytes derived from human pluripotent stem cells is an important indicator of drug reactivity in the evaluation of pro-arrhythmia and cardiotoxicity (A. M. da Rocha et al., Sci. Reports 7:13834, 2017). Mature differentiation of human pluripotent stem cells into cardiomyocytes and differentiation by cardiomyocyte type have been studied using hormones and the like, such as thyroid hormone or glucocorticoid (X. Yang et al., J. Mol. Cell Cardiol. 72:296, 2014; E. A. Rog-Zielinska et al., Cell Death Differ. 1; 22:1106, 2015), or studies have reported the promotion of differentiation into ventricular cardiomyocytes expressing cardiac troponin I (cTnI) as a mature cardiomyocyte marker and myosin light chain 2v (MLC2v) using Neuregulin-1β (O. Iglesias-Garcia et al., Stem Cells Dev. 24:484, 2015) or a low-molecular-weight compound IWR-1 (I. Karakikes et al., Stem Cells Transl. Med. 3:18, 2014). In addition, differentiation studies by cardiomyocyte type, which promote differentiation into atrial cardiomyocytes by inducing the expression of COUP-TFI and COUP-TFII signal regulators (S. P. Wu et al., Dev. Cell 25:417, 2013; H. D. Devalla et al., EMBO Mol. Med. 7:394, 2015), or which promote differentiation into nodal cardiomyocytes by inhibiting NRG-1p/ErbB signaling (Circ. Res. 107:776, 2010), are being conducted, but the maturation state of cardiomyocytes is not high, either structurally or functionally.

For acute myocardial infarction, it is clinically important to develop early diagnosis technology capable of quickly and accurately diagnosing acute myocardial infarction after the onset thereof. High-sensitivity cardiac troponin I (hs-cTnI), which is used as a representative diagnostic biomarker, has higher specificity and a longer half-life in the blood than other cardiovascular disease markers, such as creatinine kinase-MB isoform (CK-MB), myoglobin, and the like, and is thus proven to be useful as a prognostic factor for long-term elevation (A. Thomson et. al., Lancet 375:1536, 2010). In particular, the Troponin I (TnI) molecule constituting the sarcomere exists as an ssTroponin I (ssTnI) isoform in the fetus, but as development progresses, it is converted into cTnI, and two troponin isomers are expressed in newborns. However, it has been reported that only cTnI is expressed in adult cardiac cells (F. B. Bedada et al., Stem Cell Reports 3:594, 2014). Recently, studies using human-stem-cell-derived immature cardiomyocytes for in-vitro cardiovascular disease modeling have been attempted, but no study results have reported the detection of cTnI, which is known to be expressed in adult cardiomyocytes as a representative acute myocardial infarction marker. Hence, there is a need for in-vitro myocardial ischemia or acute myocardial infarction modeling studies after differentiation of human pluripotent stem cells into mature cardiomyocytes expressing cTnI and MLC2v.

Accordingly, the present inventors have endeavored to produce mature ventricular cardiomyocytes usable for an in-vitro acute myocardial infarction disease modeling system, and have ascertained that fibroblast growth factor 4 (FGF4) increases type-specific differentiation and mature differentiation of stem cells into cardiomyocytes, and a combination of FGF4 and ascorbic acid (AA) greatly increases differentiation into ventricular type cardiomyocytes, thereby producing mature ventricular cardiomyocytes from stem cells, and also that the mature ventricular cardiomyocytes thus produced are cultured under hypoxic (2% O₂) conditions similar to those of acute myocardial infarction, so acute-myocardial-infarction-specific markers (cTnI, myoglobin, CK-MB) secreted into the culture medium by dead cardiomyocytes may be detected simultaneously, thus culminating in the present invention.

[Disclosure]

It is an object of the present invention to provide a method of inducing differentiation of stem cells into cardiomyocytes comprising culturing stem cells in a medium containing FGF4 and ascorbic acid in order to produce mature ventricular cardiomyocytes usable for cardiovascular disease models and drug toxicity tests.

It is another object of the present invention to provide a cardiovascular disease model constructed by subjecting mature ventricular cardiomyocytes differentiated from stem cells to culture under hypoxic conditions, and a method of screening a therapeutic agent for acute myocardial infarction using the constructed cardiovascular disease cell model.

In order to accomplish the above objects, the present invention provides a method of inducing differentiation of stem cells into cardiomyocytes comprising culturing stem cells in a medium containing FGF4.

In addition, the present invention provides a method of constructing a cardiovascular disease cell model comprising i) differentiating stem cells into cardiomyocytes by culturing the stem cells in a medium containing FGF4 and ii) culturing the differentiated cardiomyocytes under hypoxic conditions.

In addition, the present invention provides a method of screening a therapeutic agent for acute myocardial infarction disease comprising i) treating a cardiovascular disease cell model constructed through the method described above with a candidate for a therapeutic agent for acute myocardial infarction, and ii) selecting the candidate as a therapeutic agent for acute myocardial infarction disease when secretion of an acute-myocardial-infarction-specific marker is decreased compared to a cardiovascular disease cell model not treated with the candidate.

In addition, the present invention provides a composition for inducing differentiation of stem cells into cardiomyocytes containing FGF4 as an active ingredient.

In addition, the present invention provides a kit for constructing a cardiovascular disease cell model comprising the composition for inducing differentiation.

### DESCRIPTION OF DRAWINGS

FIG. 1a schematically shows a process for differentiation of human embryonic stem cells (BG01 cell line) into cardiomyocytes by treatment with 10 ng/ml FGF2, 10 ng/ml FGF4, 10 ng/ml FGF10, 200 μg/ml ascorbic acid, and 10 ng/ml FGF4+200 μg/ml ascorbic acid;

FIG. 1B shows optical microscope images confirming changes in a structurally thickened cell morphology in the groups treated with 10 ng/ml FGF2, 10 ng/ml FGF4, 10 ng/ml FGF10, 200 μg/ml ascorbic acid, and 10 ng/ml FGF4+200 μg/ml ascorbic acid compared to the control group (a scale increment of 200 μm);

FIG. 2 shows the results of a comparison at the mRNA level on expression of a cardiomyocyte marker (cTnT), mature cardiomyocyte marker (cTnI), ventricular cardiomyocyte marker (MLC2v), atrial cardiomyocyte markers (MLC2a, ANP), nodal cardiomyocyte markers (HCN4, TBX18), vascular smooth muscle cell markers (SMA, SM22), and vascular endothelial cell marker (CD31) after treatment with 10 ng/ml FGF2 for 10 days from the 5th day to the 15^{th} day during the differentiation of human embryonic stem cells (BG01) into cardiomyocytes;

FIG. 3 shows the results of a comparison at the mRNA level on expression of the cardiomyocyte marker (cTnT), mature cardiomyocyte marker (cTnI), ventricular cardiomyocyte marker (MLC2v), atrial cardiomyocyte markers (MLC2a, ANP), nodal cardiomyocyte markers (HCN4, TBX18), vascular smooth muscle cell markers (SMA, SM22), and vascular endothelial cell marker (CD31) after treatment with 10 ng/ml FGF10 for 10 days from the 5^{th} day to the 15^{th} day during the differentiation of human embryonic stem cells (BG01) into cardiomyocytes;

FIG. 4 shows the results of a comparison at the mRNA level on expression of the cardiomyocyte marker (cTnT), mature cardiomyocyte marker (cTnI), ventricular cardiomyocyte marker (MLC2v), atrial cardiomyocyte markers (MLC2a, ANP), nodal cardiomyocyte markers (HCN4, TBX18), vascular smooth muscle cell markers (SMA, SM22), and vascular endothelial cell marker (CD31) after treatment with 200 μg/ml ascorbic acid for 10 days from the 5^{th} day to the 15^{th} day during the differentiation of human embryonic stem cells (BG01) into cardiomyocytes;

FIG. 5 shows the results of a comparison at the mRNA level on expression of the cardiomyocyte marker (cTnT), mature cardiomyocyte marker (cTnI), ventricular cardiomyocyte marker (MLC2v), atrial cardiomyocyte markers (MLC2a, ANP), nodal cardiomyocyte markers (HCN4, TBX18), vascular smooth muscle cell markers (SMA, SM22), and vascular endothelial cell marker (CD31) in the control group, the group treated with 10 ng/ml FGF4, and the group co-treated with 10 ng/ml FGF4+200 μg/ml ascorbic acid for 10 days from the 5^{th} day to the 15^{th} day during the differentiation of human embryonic stem cells (BG01) into cardiomyocytes;

FIG. 6 shows the results of analysis performed using polymerase chain reaction on the difference in gene expression of a ventricular type marker (MLC2v), atrial cardiomyocyte marker (MLC2a), nodal cardiomyocyte marker (TBX18), and vascular smooth muscle cell marker (SMA) depending on the concentration of FGF4 for 10 days from the 5^{th} day to the 15^{th} day during the differentiation of human embryonic stem cells (BG01) into cardiomyocytes;

FIG. 7 shows the results of comparison and analysis through immunostaining on the difference in the expression of cardiomyocyte markers (cTnT, α-actinin), ventricular cardiomyocyte marker (MLC2v), and atrial cardiomyocyte marker (MLC2a) in the control group, the group treated with 10 ng/ml FGF4, and the group co-treated with 10 ng/ml FGF4+200 μg/ml ascorbic acid for 10 days from the 5^{th} day to the 15^{th} day during the differentiation of human embryonic stem cells (BG01) into cardiomyocytes (a scale increment of 100 μm);

FIG. 7a shows the results of comparison and analysis through immunostaining on simultaneous expression of the cardiomyocyte marker cTnT and the ventricular cardiomyocyte marker MLC2v;

FIG. 7b shows the results of comparison and analysis through immunostaining on simultaneous expression of α-actinin as the cardiomyocyte marker and the ventricular cardiomyocyte marker MLC2v;

FIG. 7c shows the results of comparison and analysis through immunostaining on simultaneous expression of the cardiomyocyte marker cTnT and the atrial cardiomyocyte marker MLC2a;

FIG. 8a shows a graph for one or more beating wavelengths by setting spots having a certain area in the beating cardiomyocytes in each video and measuring the beating intensity within the area after differentiation of human embryonic stem cells (BG01 cell line) into cardiomyocytes in the control group and the group co-treated with 10 ng/ml FGF4+200 μg/ml ascorbic acid;

FIG. 8b shows the results of quantification of the difference in the beating interval in the control group and the group co-treated with 10 ng/ml FGF4+200 μg/ml ascorbic acid;

FIG. 9a schematically shows a culture process in chronological order under normoxic (21% O₂) and hypoxic (2% O₂) conditions after inducing mature differentiation of human embryonic stem cells (BG01 cell line) into cardiomyocytes by treatment with 10 ng/ml FGF4+200 μg/ml ascorbic acid;

FIG. 9b schematically shows detection of the expression of acute myocardial infarction markers in a conditioned medium recovered at two-day intervals from the 11^{th} day to the 21st day after differentiation from each of the immature cardiomyocytes of the control group and the mature cardiomyocytes of the group treated with 10 ng/ml FGF4+200 μg/ml ascorbic acid produced according to the conditions of FIG. 9a;

FIG. 10a shows detection of the acute myocardial infarction marker cTnI, FIG. 10b shows detection of the acute myocardial infarction marker CK-MB, and FIG. 10c shows detection of myoglobin, serving as an acute myocardial infarction marker, using, as a sample, the conditioned medium recovered from each of the control group and the group treated with 10 ng/ml FGF4+200 μg/ml ascorbic acid;

FIG. 11a schematically shows the selection and verification of genes showing differences in expression through next-generation sequencing (NGS) and polymerase chain reaction after mature cardiomyocytes produced by treatment with 10 ng/ml FGF4+200 μg/ml ascorbic acid for 10 days from the 5^{th} day to the 15^{th} day during the differentiation of human embryonic stem cells (BG01) into cardiomyocytes are cultured for 24 hours under normoxic (21% O₂) and hypoxic (2% O₂) conditions;

FIG. 11b shows the results of comparison and analysis of genes, the expression difference of which is increased or decreased by a factor of at least two, through next-generation sequencing after mature cardiomyocytes produced according to the conditions of FIG. 11a are cultured for 24 hours under normoxic and hypoxic conditions;

FIG. 12a shows the selection of hypoxia-induced cellular response genes through next-generation sequencing after mature cardiomyocytes produced by treatment with 10 ng/ml FGF4+200 μg/ml ascorbic acid for 10 days from the 5^{th} day to the 15^{th} day during the differentiation of human embryonic stem cells (BG01) into cardiomyocytes are cultured for 24 hours under hypoxic conditions;

FIG. 12b shows the results of verification through polymerase chain reaction on changes in expression of the hypoxia-induced cellular response genes identified through next-generation sequencing;

FIG. 13a shows the selection of hypoxia-inducible factor 1 (HIF-1) signaling genes through next-generation sequencing after mature cardiomyocytes produced by treatment with 10 ng/ml FGF4+200 μg/ml ascorbic acid for 10 days from the 5^{th} day to the 15^{th} day during the differentiation of human embryonic stem cells (BG01) into cardiomyocytes are cultured for 24 hours under hypoxic conditions;

FIG. 13b shows the results of verification through polymerase chain reaction on changes in expression of the HIF-1 signaling genes identified through next-generation sequencing;

FIG. 14 shows the selection of hypoxia-induced apoptotic genes through next-generation sequencing after mature cardiomyocytes produced by treatment with 10 ng/ml FGF4+200 μg/ml ascorbic acid for 10 days from the 5^{th} day to the 15^{th} day during the differentiation of human embryonic stem cells (BG01) into cardiomyocytes are cultured for 24 hours under hypoxic conditions;

FIG. 15a shows the results of comparison and analysis through immunostaining on simultaneous expression of cTnT as the cardiomyocyte marker and C-cas3 after mature cardiomyocytes produced by treatment with 10 ng/ml FGF4+200 μg/ml ascorbic acid for 10 days from the 5^{th} day to the 15^{th} day during the differentiation of human embryonic stem cells (BG01) into cardiomyocytes are cultured for 24 hours under normoxic and hypoxic conditions (a scale increment of 100 μm); and

FIG. 15b shows the results of comparison and analysis through immunostaining on induction of cleaved-Caspase-3 (C-cas3) as an apoptosis marker under hypoxic conditions through simultaneous expression of α-actinin as the cardiomyocyte marker and C-cas3 (a scale increment of 100 μm).

### MODE FOR INVENTION

Unless otherwise defined, all technical and scientific terms used herein have the same meanings as those typically understood by those skilled in the art to which the present invention belongs. Generally, the nomenclature used herein is well known in the art and is typical.

In the present invention, when differentiating stem cells into cardiomyocytes using FGF4 or FGF4+ascorbic acid, the rate of differentiation into cardiomyocytes and the maturation state were increased. In addition, differentiation by cardiomyocyte type was confirmed by analyzing the expression of atrial or ventricular cell markers, and when stem cells were differentiated in a medium containing FGF4 or FGF4+ascorbic acid under monolayer culture conditions, type-specific differentiation and differentiation into mature cardiomyocytes were enhanced compared to the control group. In particular, the mature ventricular cardiomyocytes thus differentiated are suitable for use as an in-vitro cell model for cardiovascular disease because expression of the mature cardiomyocyte marker cTnI and the ventricular cardiomyocyte marker MLC2v is increased, expression of the vascular smooth muscle cell marker and the nodal cardiomyocyte marker is decreased, and heartbeat is uniform.

In an embodiment of the present invention, when differentiating stem cells into cardiomyocytes using 10 ng/ml FGF2, 10 ng/ml FGF4, 10 ng/ml FGF10, 200 μg/ml ascorbic acid, and 10 ng/ml FGF4+200 μg/ml ascorbic acid, the stem cells were formed in a thickened structure in the group treated with 10 ng/ml FGF4 or with 10 ng/ml FGF4+200 μg/ml ascorbic acid, and beating cardiomyocytes were observed 24 hours earlier.

In another embodiment of the present invention, the expression of a cardiomyocyte marker (cTnT), mature cardiomyocyte marker (cTnI), ventricular cardiomyocyte marker (MLC2v), atrial cardiomyocyte markers (MLC2a, ANP), nodal cardiomyocyte markers (HCN4, TBX18), vascular smooth muscle cell markers (SMA, SM22), and vascular endothelial cell marker (CD31) was investigated upon treatment with 10 ng/ml FGF2, 10 ng/ml FGF4, 10 ng/ml FGF10, 200 μg/ml ascorbic acid, and 10 ng/ml FGF4+200 μg/ml ascorbic acid. As a result, expression of the mature cardiomyocyte marker (cTnI), ventricular cardiomyocyte marker (MLC2v), and atrial cardiomyocyte marker (MLC2a, ANP) was significantly increased upon treatment with 10 ng/ml FGF4, and expression of the ventricular cardiomyocyte marker (MLC2v) was significantly increased in the group co-treated with 10 ng/ml FGF4+200 μg/ml ascorbic acid compared to the control group and the groups treated with 10 ng/ml FGF, whereas expression of the vascular smooth muscle cell markers (SMA, SM22) and nodal cardiomyocyte markers (HCN4, TBX18) was significantly decreased.

In still another embodiment of the present invention, based on results confirming gene expression of the ventricular cardiomyocyte marker (MLC2v), atrial cardiomyocyte marker (MLC2a), nodal cardiomyocyte marker (TBX18), and vascular smooth muscle cell marker (SMA) in the FGF4 concentration range of 5 to 100 ng/ml, the gene expression of the ventricular cardiomyocyte type marker (MLC2v) and the atrial cardiomyocyte type marker (MLC2a) started to increase in the group treated with 5 ng/ml FGF4, the gene expression was increased the most at FGF4 concentrations of 10 ng/ml and 25 ng/ml, and the gene expression was also observed to increase even at 50 ng/ml FGF4.

Accordingly, an aspect of the present invention pertains to a method of inducing the differentiation of stem cells into cardiomyocytes comprising culturing stem cells in a medium containing FGF4.

In the present invention, the concentration of FGF4 is preferably 5 ng/ml to 50 ng/ml, and more preferably 10 ng/ml to 25 ng/ml, but is not limited thereto.

In the present invention, it is preferable that the medium further contain ascorbic acid, and the concentration of ascorbic acid is preferably 100 μg/ml to 300 μg/ml, and more preferably 200 μg/ml, but is not limited thereto.

As used herein, the term “stem cell” refers to pluripotent stem cells comprising embryonic stem cells and induced pluripotent stem cells derived from the inner cell mass in the blastocyst of an early-developmental-stage embryo having pluripotency. The stem cells are stem cells obtained from one or more species among rodents, comprising mice and rats, and primates, comprising gorillas and humans, but are not limited thereto.

In the present invention, the stem cells are preferably embryonic stem cells, induced pluripotent stem cells, or adult stem cells, but are not limited thereto.

Also, in an embodiment of the present invention, the embryonic stem cells or induced pluripotent stem cells are preferably inoculated at 2.5×10⁵ to 2×10⁶ cells per well of a 6-well plate, but the present invention is not limited thereto.

As used herein, the term “differentiation” means that cells change to the level of an individual cell or a complex of specific cells or tissues having a special function during cell division, proliferation, and growth.

Since the use of mature cardiomyocytes for disease modeling and drug toxicity evaluation enables evaluation with improved efficacy and safety, studies for differentiation of immature cardiomyocytes comprising atrial, ventricular and nodal cardiomyocyte types into mature cardiomyocytes and differentiation by cardiomyocyte type are needed. The maturation state of cardiomyocytes derived from human pluripotent stem cells is an important indicator of drug reactivity when evaluating pro-arrhythmia and cardiotoxicity.

In the present invention, the cardiomyocytes may be mature ventricular cells, and the cardiomyocytes preferably express the mature cardiomyocyte marker cTnI and the ventricular cardiomyocyte marker MLC2v, but the present invention is not limited thereto.

The mature ventricular cardiomyocytes differentiated by culturing stem cells in a medium containing FGF4 and ascorbic acid according to the present invention are suitable for use as a cardiovascular disease cell model because the expression of atrial and ventricular type markers is increased and the expression of nodal and vascular smooth muscle cell markers is decreased.

In an embodiment of the present invention, the beating of the cardiomyocytes was synchronized in the group treated with FGF4+ascorbic acid, whereas the control group exhibited different beating of cardiomyocytes.

In the present invention, the cardiomyocytes may be beating cells.

In an embodiment of the present invention, a conditioned medium was recovered from each of the control group and the group treated with FGF4+ascorbic acid cultured under normoxic (21% O₂) and hypoxic (2% O₂) conditions from the 11^{th} day to the 21^{st} day after differentiation into cardiomyocytes, after which the expression of acute myocardial infarction markers (cTnI, myoglobin, CK-MB) was compared and evaluated using a PATHFAST instrument (Mitsubishi), indicating that the expression of the acute myocardial infarction markers was induced under hypoxic conditions. In particular, expression of acute myocardial infarction markers known to be expressed in mature cardiomyocytes was higher in the group treated with FGF4+ascorbic acid than in the control group.

In another embodiment of the present invention, the expression of hypoxia-induced cellular response genes, hypoxia-inducible factor 1 (HIF-1) signaling genes, and hypoxia-induced apoptotic genes showed a significant difference through next-generation sequencing after 24 hours of hypoxic culture of mature cardiomyocytes treated with FGF4+ascorbic acid, and changes in the expression of genes selected through next-generation sequencing were verified again through polymerase chain reaction.

In another embodiment of the present invention, induction of cleaved-Caspase-3 (C-cas3) as an apoptosis marker under hypoxic conditions after 24 hours of hypoxic culture of mature cardiomyocytes treated with FGF4+ascorbic acid was confirmed through immunostaining.

Accordingly, another aspect of the present invention pertains to a method of constructing a cardiovascular disease cell model comprising i) differentiating stem cells into cardiomyocytes by culturing the stem cells in a medium containing FGF4 and ii) culturing the differentiated cardiomyocytes under hypoxic conditions.

In the present invention, the concentration of FGF4 is preferably 5 ng/ml to 50 ng/ml, and more preferably 10 ng/ml to 25 ng/ml, but is not limited thereto.

In the present invention, the medium in step i) preferably further contains ascorbic acid, and the concentration of ascorbic acid is preferably 100 μg/ml to 300 μg/ml, and more preferably 200 μg/ml, but the present invention is not limited thereto.

In the present invention, the cardiovascular disease is preferably acute coronary syndrome (ACS), more preferably acute myocardial infarction (AMI) or unstable angina, and most preferably acute myocardial infarction, but is not limited thereto.

In the present invention, the stem cells are preferably embryonic stem cells, induced pluripotent stem cells, or adult stem cells, but are not limited thereto.

Also, in an embodiment of the present invention, the embryonic stem cells or induced pluripotent stem cells are preferably inoculated at 2.5×10⁵ to 2×10⁶ cells per well of a 6-well plate, but the present invention is not limited thereto.

In the present invention, the hypoxia is preferably an oxygen partial pressure of 1% to 5%, but the present invention is not limited thereto.

Recently, a study reported the detection of myocardial infarction biomarkers cTnT, FABP3, etc. in a culture medium as a result of doxorubicin treatment using, as a model, cardiomyocytes derived from human pluripotent stem cells (H. Andersson et al., J. Biotechnol. 150:175, 2010).

In an embodiment of the present invention, it was suggested that, after inducing apoptosis of cardiomyocytes through hypoxic (2% O₂) culture of mature differentiated cardiomyocytes, a representative acute myocardial infarction marker cTnI known to be expressed in mature cardiomyocytes and other acute myocardial infarction markers (myoglobin, CK-MB), which were secreted from damaged cardiomyocytes, were detected in a culture medium, and thus the mature differentiated cardiomyocytes were useful in an in-vitro acute myocardial infarction disease modeling system.

In another example of the present invention, the expression of the ventricular cardiomyocyte marker MLC2v was observed to increase in the group treated with FGF4 or with FGF4+ascorbic acid compared to the control group.

Therefore, still another aspect of the present invention pertains to a method of screening a therapeutic agent for acute myocardial infarction disease comprising i) treating a cardiovascular disease cell model constructed through the method described above with a candidate for a therapeutic agent for acute myocardial infarction and ii) selecting the candidate as a therapeutic agent for acute myocardial infarction disease when secretion of an acute-myocardial-infarction-specific marker is decreased compared to a cardiovascular disease cell model not treated with the candidate.

In the present invention, the acute-myocardial-infarction-specific marker may be cTnI (cardiac troponin I), myoglobin, or CK-MB (creatinine kinase-MB isoform).

In the present invention, secretion of the acute-myocardial-infarction-specific marker is preferably measured in the conditioned medium of the cell model, but the present invention is not limited thereto.

In the present invention, in order to solve problems in which the cardiomyocytes differentiated from human embryonic or induced pluripotent stem cells structurally or functionally show the characteristics of embryonic cardiomyocytes and immature cardiomyocytes and are thus unsuitable for disease modeling and drug toxicity testing, FGF4 or FGF4+ascorbic acid was used in the step of inducing differentiation of stem cells.

Yet another aspect of the present invention pertains to a composition for inducing differentiation of stem cells into cardiomyocytes containing FGF4 as an active ingredient.

Still yet another aspect of the present invention pertains to a kit for constructing a cardiovascular disease cell model comprising the composition for inducing differentiation.

### EXAMPLES

A better understanding of the present invention may be obtained through the following examples. These examples are merely set forth to illustrate the present invention, and are not to be construed as limiting the scope of the present invention, as will be apparent to those skilled in the art.

### Example 1: Confirmation of Morphology and Beating of Cardiomyocytes Differentiated from Stem Cells

In order to increase the differentiation efficiency of stem cells into cardiomyocytes and to improve differentiation by cardiomyocyte type and mature differentiation, conditions for monolayer differentiation of human embryonic stem cells (BG01 cell line) into cardiomyocytes were established.

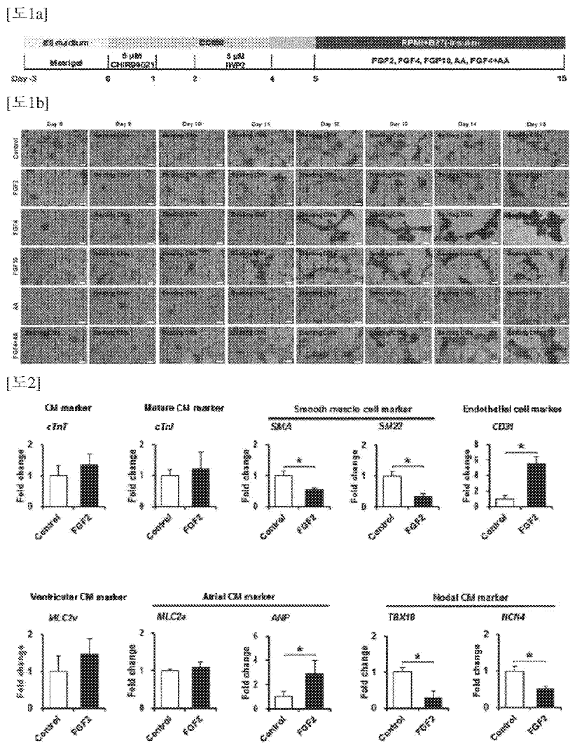

An E8 culture medium (STEMCELL Technologies) containing a 2 μM ROCK inhibitor (thiazovivin) was added to a 6-well culture plate coated with Matrigel, and embryonic stem cells dissociated into single cells using Accutase were seeded at 2.5×10⁵ cells per well. Culture was carried out for about 3 days, and the E8 culture medium was exchanged until confluence of the 6-well culture plate with the cells. When confluence of the 6-well plate with the human embryonic stem cells was achieved, CHIR99021 (Sigma-Aldrich) as a GSK-3 inhibitor was added at a concentration of 6 μM to a CDM3 culture medium (containing RPMI 1640, serum albumin and ascorbic acid as three chemically verified components, P. W. Burridge et al., Nature Methods 11:855, 2014), followed by culture for 24 hours. Then, on the 2nd day of differentiation, 5 μM IWP2 (Inhibitor of Wnt Production 2) was added to the medium, followed by culture for 2 days. After addition of 10 ng/ml FGF2, 10 ng/ml FGF4, 10 ng/ml FGF10, 200 μg/ml ascorbic acid, and 10 ng/ml FGF4+200 μg/ml ascorbic acid on the 5^{th} day of differentiation, culture was carried out until the 15^{th} day of differentiation while the culture medium was exchanged every 48 hours with an RPMI+B27(-Insulin) medium (FIG. 1a).

Consequently, it was observed that, in the group treated with 10 ng/ml FGF4 or with 10 ng/ml FGF4+200 μg/ml ascorbic acid, the stem cells were formed in a thickened structure (FIG. 1B), and the groups treated with 10 ng/ml FGF2 and with 10 ng/ml FGF10 showed morphological changes similar to the control group. Moreover, the group treated with 200 μg/ml ascorbic acid showed a relatively flat morphology, unlike the agglomerated and thickened morphology observed in the control group and the groups treated with FGF. The beating cardiomyocytes were observed on the 9^{th} day of differentiation in the control group and the groups treated with 10 ng/ml FGF2, 10 ng/ml FGF4, 10 ng/ml FGF10, and 200 μg/ml ascorbic acid, but were observed from about the 8^{th} day, which was about 24 hours earlier, in the group treated with 10 ng/ml FGF4+200 μg/ml ascorbic acid (FIG. 1B).

### Example 2: Effect of Each Maturation Factor on Differentiation by Cardiomyocyte Type and Mature Differentiation

2-1: FGF2

In order to investigate the effect of FGF2 on differentiation by cardiomyocyte type, mature differentiation of cardiomyocytes, and differentiation of vascular smooth muscle cells, the expression of a cardiomyocyte marker (cTnT), mature cardiomyocyte marker (cTnI), ventricular cardiomyocyte marker (MLC2v), atrial cardiomyocyte markers (MLC2a, ANP), nodal cardiomyocyte markers (HCN4, TBX18), vascular smooth muscle cell markers (SMA, SM22), and vascular endothelial cell marker (CD31) in the control group and the group treated with 10 ng/ml FGF2 for 10 days from the 5^{th} day to the 15^{th} day during the differentiation of human embryonic stem cells (BG01) into cardiomyocytes was determined through polymerase chain reaction using primers for each cell lineage marker.

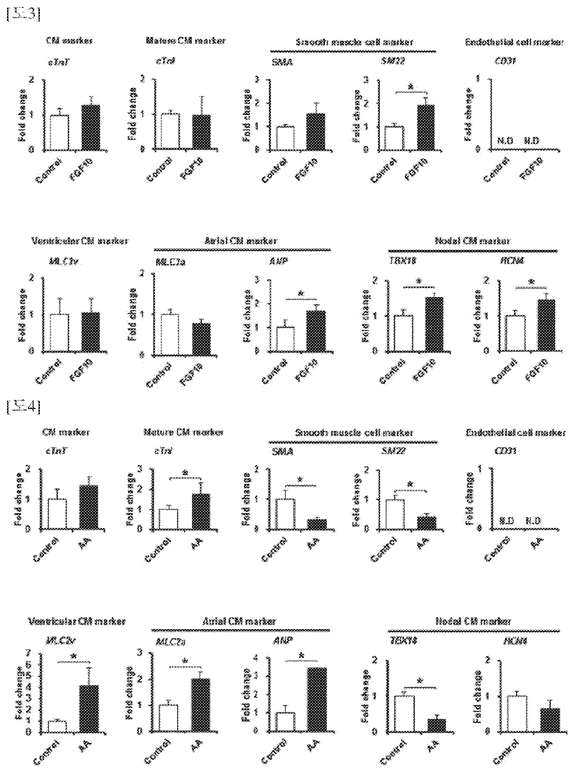

Consequently, it was confirmed that the expression of the vascular endothelial cell marker (CD31) and the expression of the atrial cardiomyocyte marker ANP were significantly increased by treatment with 10 ng/ml FGF2 on the 15^{th} day after differentiation. In contrast, the expression of the vascular smooth muscle cell markers (SMA, SM22) and the nodal cardiomyocyte markers (HCN4, TBX18) was significantly decreased compared to the control group. However, it was confirmed that there was no effect on the expression of the mature cardiomyocyte marker (cTnI) and the ventricular cardiomyocyte marker (MLC2v) (FIG. 2).

2-2: FGF10

In order to investigate the effect of FGF10 on differentiation by cardiomyocyte type, mature differentiation of cardiomyocytes, and differentiation of vascular smooth muscle cells, the expression of the cardiomyocyte marker (cTnT), mature cardiomyocyte marker (cTnI), ventricular cardiomyocyte marker (MLC2v), atrial cardiomyocyte markers (MLC2a, ANP), nodal cardiomyocyte markers (HCN4, TBX18), vascular smooth muscle cell markers (SMA, SM22), and vascular endothelial cell marker (CD31) in the control group and the group treated with 10 ng/ml FGF10 for 10 days from the 5^{th} day to the 15^{th} day during the differentiation of human embryonic stem cells (BG01) into cardiomyocytes was determined through polymerase chain reaction using primers for each cell lineage marker.

Consequently, it was confirmed that the expression of the vascular smooth muscle cell marker (SMA), the atrial cardiomyocyte marker (MLC2a), and the nodal cardiomyocyte markers (HCN4, TBX18) was significantly increased by treatment with 10 ng/ml FGF10 on the 15^{th} day after differentiation. However, it was confirmed that there was no effect on the expression of the mature cardiomyocyte marker (cTnI) and the ventricular cardiomyocyte marker (MLC2v) (FIG. 3).

2-3: Ascorbic acid

In order to investigate the effect of ascorbic acid on differentiation by cardiomyocyte type, mature differentiation of cardiomyocytes, and differentiation of vascular smooth muscle cells, the expression of the cardiomyocyte marker (cTnT), mature cardiomyocyte marker (cTnI), ventricular cardiomyocyte marker (MLC2v), atrial cardiomyocyte markers (MLC2a, ANP), nodal cardiomyocyte markers (HCN4, TBX18), vascular smooth muscle cell markers (SMA, SM22), and vascular endothelial cell marker (CD31) in the control group and the group treated with 200 μg/ml ascorbic acid for 10 days from the 5^{th} day to the 15^{th} day during the differentiation of human embryonic stem cells (BG01) into cardiomyocytes was determined through polymerase chain reaction using primers for each cell lineage marker.

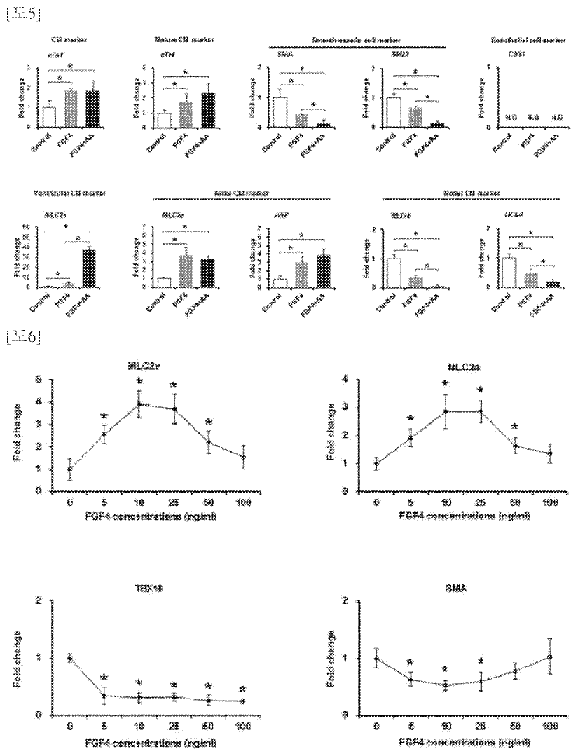

Consequently, it was confirmed that the expression of the cardiomyocyte marker (cTnT), the mature cardiomyocyte marker (cTnI), the ventricular cardiomyocyte marker (MLC2v), and the atrial cardiomyocyte markers (MLC2a, ANP) was significantly increased by treatment with 200 μg/ml ascorbic acid on the 15^{th} day after differentiation. In contrast, it was confirmed that the expression of the vascular smooth muscle cell markers (SMA, SM22) and the nodal cardiomyocyte markers (HCN4, TBX18) was significantly decreased (FIG. 4).

2-4: FGF4

In order to investigate the effect of FGF4 on differentiation by cardiomyocyte type, mature differentiation of cardiomyocytes, and differentiation of vascular smooth muscle cells, the expression of the cardiomyocyte marker (cTnT), mature cardiomyocyte marker (cTnI), ventricular cardiomyocyte marker (MLC2v), atrial cardiomyocyte markers (MLC2a, ANP), nodal cardiomyocyte markers (HCN4, TBX18), vascular smooth muscle cell markers (SMA, SM22), and vascular endothelial cell marker (CD31) in the control group and the group treated with 10 ng/ml FGF4 for 10 days from the 5^{th} day to the 15^{th} day during the differentiation of human embryonic stem cells (BG01) into cardiomyocytes was determined through polymerase chain reaction using primers for each cell lineage marker.

Consequently, it was confirmed that the expression of the mature cardiomyocyte marker (cTnI), the ventricular cardiomyocyte marker (MLC2v), and the atrial cardiomyocyte markers (MLC2a, ANP) was significantly increased by treatment with 10 ng/ml FGF4 on the 15^{th} day after differentiation. In contrast, it was confirmed that the expression of the vascular smooth muscle cell markers (SMA, SM22) and the nodal cardiomyocyte markers (HCN4, TBX18) was significantly decreased (FIG. 5).

2-5: FGF4 and Ascorbic Acid

In order to investigate the effect of co-treatment with FGF4 and ascorbic acid, which significantly increase the expression of a mature cardiomyocyte marker (cTnI) and a ventricular cardiomyocyte marker (MLC2v), on differentiation by cardiomyocyte type, mature differentiation of cardiomyocytes, and differentiation of vascular smooth muscle cells, the expression of the cardiomyocyte marker (cTnT), mature cardiomyocyte marker (cTnI), ventricular cardiomyocyte marker (MLC2v), atrial cardiomyocyte markers (MLC2a, ANP), nodal cardiomyocyte markers (HCN4, TBX18), vascular smooth muscle cell markers (SMA, SM22), and vascular endothelial cell marker (CD31) in the group co-treated with 10 ng/ml FGF4+200 μg/ml ascorbic acid for 10 days from the 5^{th} day to the 15^{th} day during the differentiation of human embryonic stem cells (BG01) into cardiomyocytes was determined through polymerase chain reaction using primers for each cell lineage marker.

Consequently, a synergistic effect in which the expression of the ventricular cardiomyocyte marker (MLC2v) was significantly increased in the group co-treated with 10 ng/ml FGF4+200 μg/ml ascorbic acid compared to the control group and the group treated with 10 ng/ml FGF4 on the 15^{th} day after differentiation was confirmed. In contrast, it was confirmed that the expression of the vascular smooth muscle cell markers (SMA, SM22) and the nodal cardiomyocyte markers (HCN4, TBX18) was significantly decreased. However, it was confirmed that the gene expression of the cardiomyocyte marker (cTnT), the mature cardiomyocyte marker (cTnI), and the atrial cardiomyocyte markers (MLC2a, ANP) did not show a significant increase or decrease between the group co-treated with 10 ng/ml FGF4+200 μg/ml ascorbic acid and the group treated with 10 ng/ml FGF4 alone (FIG. 5).

### Example 3: Concentration of FGF4 for Differentiation by Cardiomyocyte Type and Differentiation of Vascular Smooth Muscle Cells

In order to determine the appropriate concentration of FGF4 on the increase in gene expression of the ventricular cardiomyocyte marker (MLC2v), atrial cardiomyocyte marker (MLC2a), nodal cardiomyocyte marker (TBX18), and vascular smooth muscle cell marker (SMA) from human embryonic stem cells, after treatment with FGF4 at various concentrations for 10 days from the 5^{th} day to the 15^{th} day of differentiation, polymerase chain reaction was performed using primers for MLC2v, MLC2a, TBX18, and SMA.

Consequently, the gene expression of the ventricular cardiomyocyte type marker (MLC2v) and the atrial cardiomyocyte type marker (MLC2a) started to increase in the group treated with 5 ng/ml FGF4, the gene expression was the highest upon treatment with 10 ng/ml and 25 ng/ml FGF4, and the gene expression was also observed to increase at 50 ng/ml FGF4 (FIG. 6). In contrast, it was confirmed that the gene expression of the nodal cardiomyocyte marker (TBX18) was significantly decreased in all of the groups treated with 5 ng/ml FGF4, 10 ng/ml FGF4, 25 ng/ml FGF4, and 100 ng/ml FGF4. The gene expression of the vascular smooth muscle cell marker (SMA) was observed to significantly decrease upon treatment with 5 ng/ml FGF4, 10 ng/ml FGF4, and 25 ng/ml FGF4 (FIG. 6).

### Example 4: Confirmation of Differentiation by Cardiomyocyte Type and Mature Differentiation

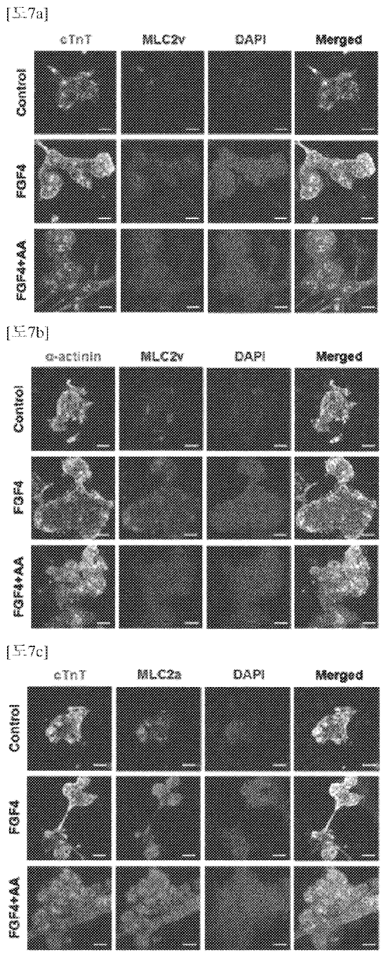

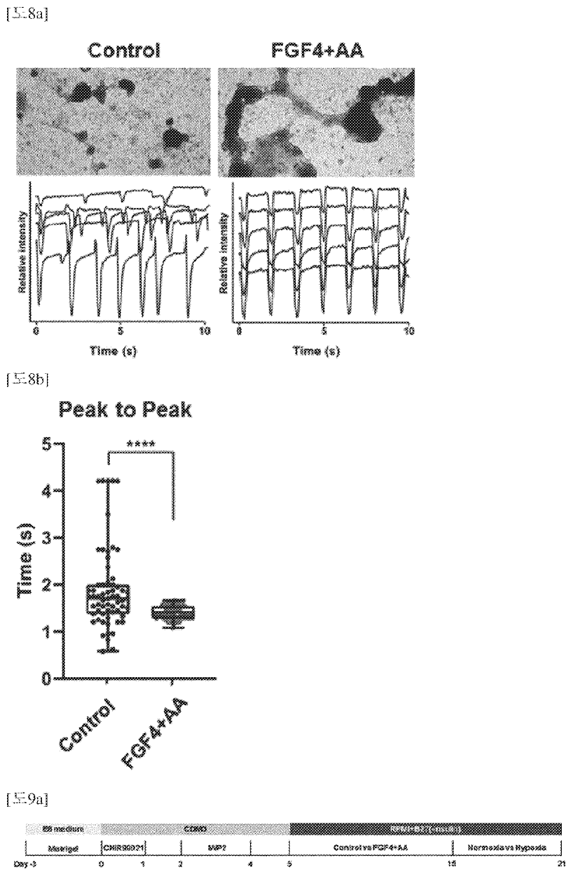

In order to investigate the effect of treatment with FGF4 alone and co-treatment with FGF4+ascorbic acid on type-specific differentiation and mature differentiation of human embryonic stem cells into cardiomyocytes, differences in expression of the cardiomyocyte marker (cTnT), ventricular cardiomyocyte marker (MLC2v), and atrial cardiomyocyte marker (MLC2a) after treatment with 10 ng/ml FGF4 or co-treatment with 10 ng/ml FGF4+200 μg/ml ascorbic acid for 10 days from the 5^{th} day to the 15^{th} day during the differentiation of human embryonic stem cells (BG01) into cardiomyocytes was confirmed through immunofluorescence staining (FIG. 7).

Consequently, it was confirmed that, in the group treated with 10 ng/ml FGF4 and the group co-treated with 10 ng/ml FGF4+200 μg/ml ascorbic acid, the cardiomyocytes showed an agglomerated and thickened morphology compared to the control group, and also that the expression of the ventricular cardiomyocyte marker MLC2v was increased in the thickened cells (FIG. 7). However, the atrial cardiomyocyte marker MLC2a showed a positive reaction in all of the control group and the group treated with 10 ng/ml FGF4 or the group co-treated with 10 ng/ml FGF4+200 μg/ml ascorbic acid due to strong expression in both immature cardiomyocytes and mature cardiomyocytes.

### Example 5: Analysis of Beating Characteristics of Cardiomyocytes

In order to investigate the effect of co-treatment with FGF4+ascorbic acid on the beating characteristics of cardiomyocytes derived from human embryonic stem cells, human embryonic stem cells were differentiated into cardiomyocytes after co-treatment with 10 ng/ml FGF4+200 μg/ml ascorbic acid for 10 days from the 5^{th} day to the 15^{th} day during the differentiation of human embryonic stem cells (BG01) into cardiomyocytes, after which five spots having a certain area were set in the beating cardiomyocytes in each video, and the beating interval (Peak to Peak) within the area was measured and then graphed (FIG. 8a).

Consequently, it was confirmed through quantitative analysis that, in the group co-treated with 10 ng/ml FGF4+200 μg/ml ascorbic acid, the beating interval of cardiomyocytes was significantly more uniform than the control group (FIG. 8b).

### Example 6: Construction of Acute Myocardial Infarction Modeling System Using Mature Cardiomyocytes

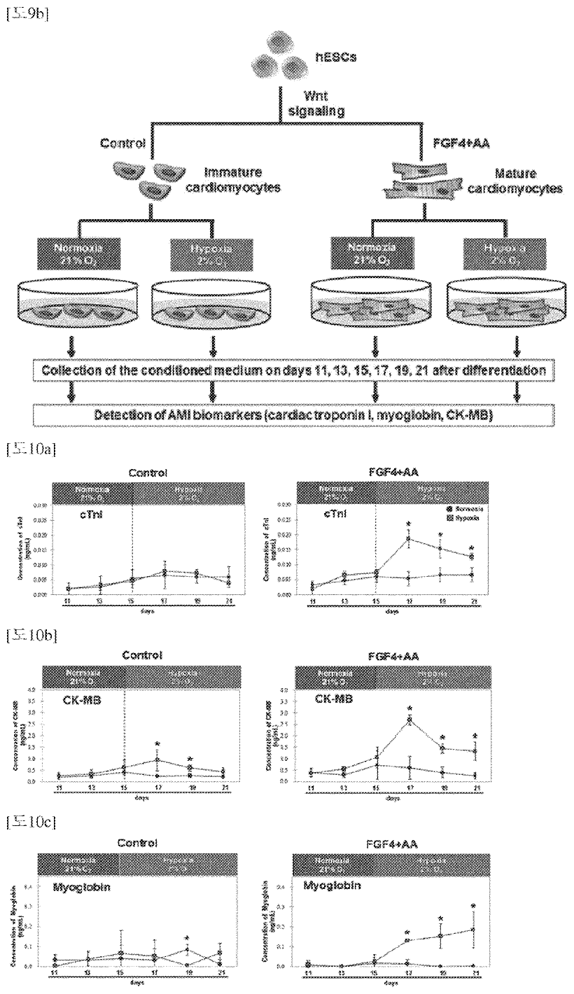

An in-vitro acute myocardial infarction disease modeling system was constructed by inducing ischemic conditions by subjecting cardiomyocytes, mature differentiated by treatment with 10 ng/ml FGF4+200 μg/ml ascorbic acid, to culture under hypoxic (2% O₂) conditions, corresponding to an environment similar to acute myocardial infarction. After inducing apoptosis of cardiomyocytes by culturing the control group and the mature cardiomyocytes under hypoxic (2% O₂) conditions, whether acute-myocardial-infarction-specific markers (cTnI, myoglobin, CK-MB) secreted from the damaged cardiomyocytes were detected in a culture medium was evaluated. An in-vitro acute myocardial infarction disease modeling system capable of comparing and evaluating acute-myocardial-infarction-specific markers in a culture medium was constructed (FIG. 9).

An E8 culture medium (STEMCELL Technologies) containing a 2 μM ROCK inhibitor (thiazovivin) was added to a 6-well culture plate coated with Matrigel, and embryonic stem cells dissociated into single cells using Accutase were seeded at 2.5×10⁵ cells per well. After culture for about 3 days, the E8 culture medium was exchanged until confluence of the 6-well culture plate with the cells. When confluence of the 6-well plate with the human embryonic stem cells was achieved, CHIR99021 (Sigma-Aldrich) as a GSK-3 inhibitor was added at a concentration of 6 μM to a CDM3 culture medium, followed by culture for 24 hours. On the 2 nd day of differentiation, 5 μM IWP2 was added to the medium, followed by culture for 2 days. On the 5^{th} day of differentiation, both 10 ng/ml FGF4 and 200 μg/ml ascorbic acid (Sigma-Aldrich) were added thereto, followed by culture until the 15^{th} day of differentiation while the culture medium was exchanged every 48 hours with an RPMI+B27(-Insulin) medium, after which the control group and the group treated with 10 ng/ml FGF4+200 μg/ml ascorbic acid (Sigma-Aldrich) were cultured until the 21^{st} day under normoxic (21% O₂) and hypoxic (2% O₂) conditions (FIG. 9a). Thereafter, the expression of acute myocardial infarction markers was detected using, as a sample, the conditioned medium recovered at two-day intervals from the 11^{th} day to the 21^{st} day after differentiation from each of the immature cardiomyocytes of the control group and the mature cardiomyocytes of the group treated with 10 ng/ml FGF4+200 μg/ml ascorbic acid. 100 μl of the conditioned medium was recovered from the cardiomyocytes of each of the control group and the group treated with 10 ng/ml FGF4+200 μg/ml ascorbic acid cultured under normoxic (21% O₂) and hypoxic (2% O₂) conditions, after which the expression of acute myocardial infarction markers was detected using a PATHFAST instrument (Mitsubishi) (FIG. 9b).

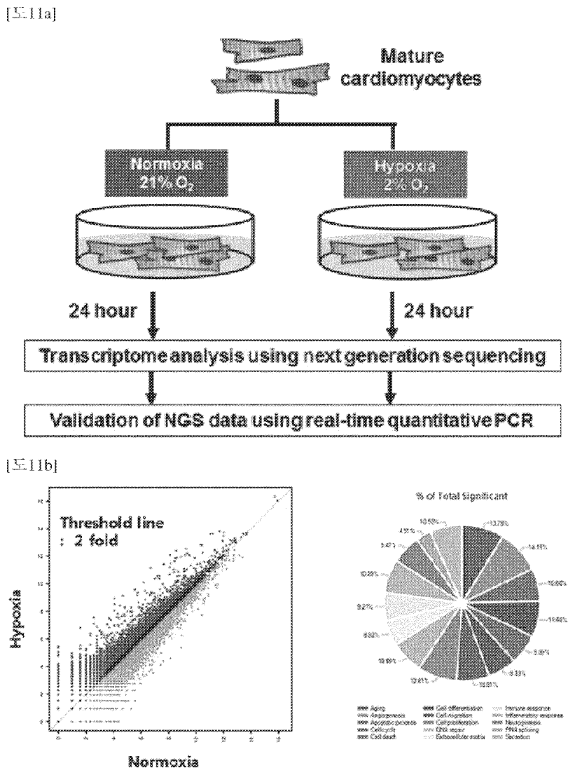

Based on the results of detection of the expression of the acute myocardial infarction markers using the PATHFAST cTnI (#1110-2000), PATHFAST Myoglobin (#1110-2001) and PATHFAST CK-MB (#1110-2002) detection kits for the PATHFAST instrument (Mitsubishi), as shown in FIG. 10, the acute myocardial infarction markers were identified in the conditioned medium of the cardiomyocytes of the group treated with 10 ng/ml FGF4+200 μg/ml ascorbic acid cultured under hypoxic (2% O₂) conditions.

### Example 7: Confirmation of Suitability of Mature Cardiomyocytes for In-Vitro Acute Myocardial Infarction Modeling

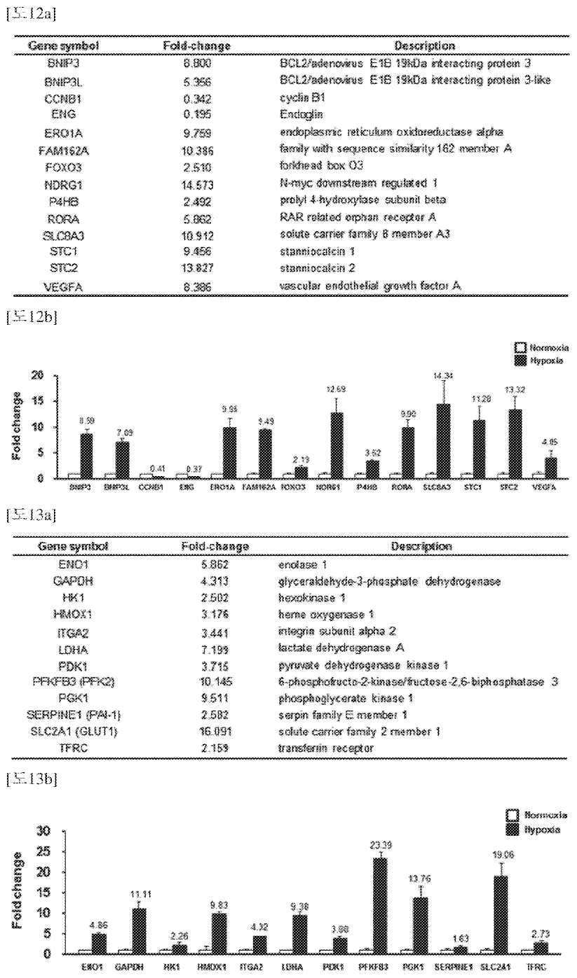

For molecular evaluation of whether the ischemic conditions induced by subjecting mature cardiomyocytes treated with 10 ng/ml FGF4+200 μg/ml ascorbic acid to culture under hypoxic (2% O₂) conditions corresponding to an environment similar to acute myocardial infarction are suitable for in-vitro acute myocardial infarction modeling, mature cardiomyocytes produced by treatment with 10 ng/ml FGF4+200 μg/ml ascorbic acid for 10 days from the 5^{th} day to the 15^{th} day during the differentiation of human embryonic stem cells (BG01) into cardiomyocytes were cultured for 24 hours under normoxic (21% O₂) and hypoxic (2% O₂) conditions, after which observed differences in gene expression were compared and analyzed through next-generation sequencing and polymerase chain reaction (FIG. 11). Moreover, genes, the expression of which was increased or decreased by a factor of at least two after treatment with 10 ng/ml FGF4+200 μg/ml ascorbic acid, were classified depending on cell function (FIG. 11b).

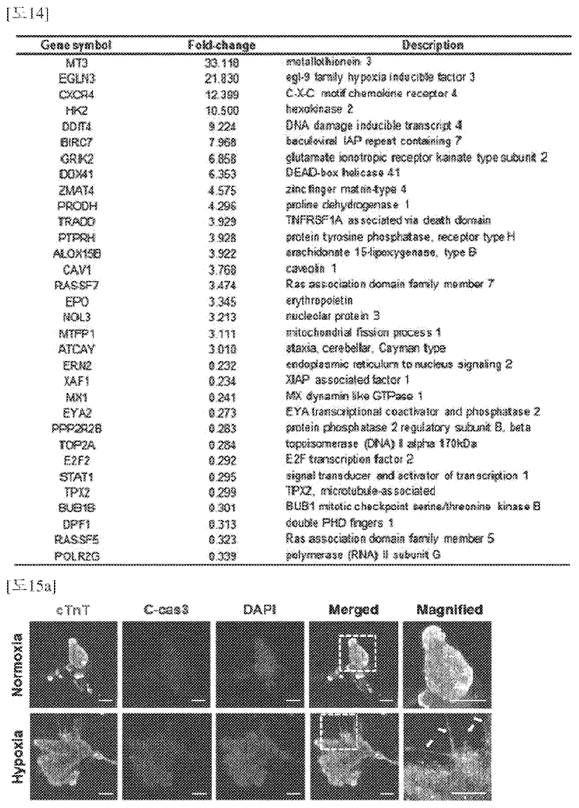

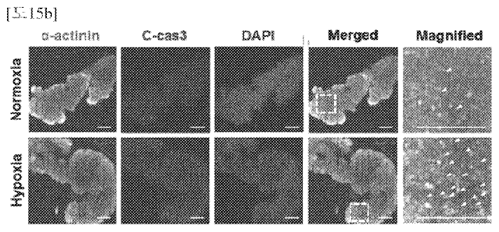

Consequently, when the mature cardiomyocytes of the group treated with 10 ng/ml FGF4+200 μg/ml ascorbic acid were cultured for 24 hours under hypoxic conditions, changes in the expression of hypoxia-induced cellular response genes (FIG. 12a), the expression of hypoxia-inducible factor 1 (HIF-1) signaling genes (FIG. 13a), and the expression of hypoxia-induced apoptotic genes (FIG. 14) were identified through next-generation sequencing. Moreover, differences in expression of hypoxia-induced cellular response genes (FIG. 12b) and HIF-1 signaling genes (FIG. 13b) selected through next-generation sequencing were verified through polymerase chain reaction.

Finally, when the mature cardiomyocytes of the group treated with 10 ng/ml FGF4+200 μg/ml ascorbic acid were cultured for 24 hours under hypoxic conditions, induction of cleaved-Caspase-3 (C-cas3) as an apoptosis marker was compared and analyzed through simultaneous expression of cTnT and α-actinin as the cardiomyocyte markers and C-cas3 using immunostaining (FIG. 15).

### INDUSTRIAL APPLICABILITY

According to the present invention, stem cells can be differentiated into mature ventricular cardiomyocytes through culture in a medium containing FGF4 and ascorbic acid, and a cardiovascular disease cell model using the differentiated mature ventricular cardiomyocytes is very useful for screening a therapeutic agent for cardiovascular disease and evaluating the toxicity of new drugs.

Although specific embodiments of the present invention have been disclosed in detail above, it will be obvious to those of ordinary skill in the art that the description is merely of preferable exemplary embodiments and is not to be construed as limiting the scope of the present invention. Therefore, the substantial scope of the present invention will be defined by the appended claims and equivalents thereof.

1. A method of inducing differentiation of stem cells into cardiomyocytes comprising culturing stem cells in a medium containing FGF4.

2. The method according to claim 1, wherein the medium further contains ascorbic acid.

3. The method according to claim 1, wherein the stem cells are embryonic stem cells, induced pluripotent stem cells, or adult stem cells.

4. The method according to claim 1, wherein the cardiomyocytes are mature ventricular cells.

5. The method according to claim 1, wherein the cardiomyocytes express a mature cardiomyocyte marker cTnI and a ventricular cardiomyocyte marker MLC2v.

6. The method according to claim 1, wherein the cardiomyocytes are beating cells.

7. A method of constructing a cardiovascular disease cell model, comprising:
i) differentiating stem cells into cardiomyocytes by culturing the stem cells in a medium containing FGF4; and
ii) culturing the differentiated cardiomyocytes under hypoxia.

8. The method according to claim 7, wherein the medium in step i) further contains ascorbic acid.

9. The method according to claim 7, wherein the cardiovascular disease is acute myocardial infarction.

10. The method according to claim 7, wherein the stem cells are embryonic stem cells, induced pluripotent stem cells, or adult stem cells.

11. The method according to claim 7, wherein the cardiomyocytes are mature ventricular cells.

12. The method according to claim 7, wherein the cardiomyocytes express a mature cardiomyocyte marker cTnI and a ventricular cardiomyocyte marker MLC2v.

13. The method according to claim 7, wherein the cardiomyocytes are beating cells.

14. The method according to claim 7, wherein the hypoxia is an oxygen partial pressure of 1% to 5%.

15. A method of screening a therapeutic agent for acute myocardial infarction disease, comprising:
i) treating a cardiovascular disease cell model constructed through the method according to claim 6 with a candidate for a therapeutic agent for acute myocardial infarction; and
ii) selecting the candidate as a therapeutic agent for acute myocardial infarction disease when secretion of an acute-myocardial-infarction-specific marker is decreased compared to a cardiovascular disease cell model not treated with the candidate.

16. The method according to claim 15, wherein the acute-myocardial-infarction-specific marker is cTnI (cardiac troponin I), myoglobin, or CK-MB (creatinine kinase-MB isoform).

17. The method according to claim 15, wherein the secretion of the acute-myocardial-infarction-specific marker is measured in a conditioned medium of the cell model.

18. A composition for inducing differentiation of stem cells into cardiomyocytes containing FGF4 as an active ingredient.

19. The composition according to claim 18, further containing ascorbic acid.

20. The composition according to claim 18, wherein the stem cells are embryonic stem cells, induced pluripotent stem cells, or adult stem cells.

21. The composition according to claim 18, wherein the cardiomyocytes are mature ventricular cells.

22. The composition according to claim 18, wherein the cardiomyocytes express a mature cardiomyocyte marker cTnI and a ventricular cardiomyocyte marker MLC2v.

23. The composition according to claim 18, wherein the cardiomyocytes are beating cells.

24. A kit for constructing a cardiovascular disease cell model comprising the composition according to claim 18.
